(19) United States
(12) Patent Application Publication
(10) Pub. No.: US 2022/0062062 A1
(43) Pub. Date: Mar. 3, 2022

(54) Healing Tape

(71) Applicant: ARTHUR H. KRUGLER, Grass Valley, CA (US)

(72) Inventor: ARTHUR H. KRUGLER, Grass Valley, CA (US)

(21) Appl. No.: 16/873,656

(22) Filed: Jun. 1, 2020

(51) Int. Cl. A61F 13/02; A61F 15/00

(52) U.S. Cl. CPC A61F 13/0243; A61F 2013/0054; A61F 15/002

(57) ABSTRACT

Disclosed is an adhesive wound healing tape comprised of a water vapor permeable backing and adhesive that can be applied over a wound and remain in place throughout the healing process. Preferably, the tape is applied over a wound without gauze bandage or non-stick gauze material. In this way, the adhesive can bond to the skin on opposing sides of a cut to secure the cut skin from movement and also compress opposing sides of the cut into one another to promote faster healing with a less visible and often invisible scar as compared to typical adhesive bandages with a gauze or non-stick gauze material over the cut or wound. The healing tape is also useful for promoting the healing of scrapes and sores, especially as can be had for elderly people with fragile skin. The healing tape performs the function of sutures to close a wound but without the scaring and infection probability of typical sutures or stitches as they are called.

Another type of skin wound is a torn flap. Skin, especially for elders, can be snagged and torn, producing a triangular ‘flap’ with a 60 degree to 90 degree apex. Edges of the flap can be pulled back to be in contact with normal skin and the healing tape applied for scarless healing.

Wounds which remove an area of skin can also be protected with an application of the healing tape. The missing skin is replaced with natural skin, without using additional scaffolding type skin. Healing time for a torn flap is longer than for cuts depending on the dimensions of the missing skin.

### BACKGROUN—PRIOR ART

The following is a tabulation of some prior art references that appear relevant.

| Pat. No. | Issue Date | Patentee |
|---|---|---|
| 3,971,384 | Jul. 27, 1976 | Hasson |
| 4,612,230 | Sep. 16, 1986 | Liland, et al. |
| 4,467,805 | Aug. 28, 1984 | Fukada |
| 5,009,663 | Apr. 23, 1991 | Broome |
| 4,999,235 | Mar. 12, 1991 | Lunn, et al. |
| 8,511,470 | Aug. 20, 2013 | Grossman |
| 6,140,549 | Oct 313, 2000 | Pompei, Jr. |
| 6,124,522 | Sep. 26, 2000 | Schroeder |
| 5,480,377 | Jan. 2, 1996 | Cartmell, et al. |
| 10,322,206 | Jun. 18, 2019 | Pins, et al. |
| 9,572,580 | Feb. 21, 2017 | Barcroft, et al. |
| 9,572,580 | Feb. 21, 2017 | Sargeant, et al. |
| 9,332,991 | May 10, 2016 | Pereira, et al. |
| 8,518,077 | Aug. 27, 2013 | O'Malley, et al. |
| 7,892,256 | Feb. 22, 2011 | Grafton, et al. |
| 5,733,270 | Mar. 31, 1998 | Ling, et al. |
| 9,180,056 | Nov. 10, 2015 | Harvard ? |

Note also; Inventor of “Healing Tape”, Arthur Krugler discussed using tape to heal cuts (only) at his website seek-ask-knock.com. This application covers more information for potential users.

### BACKGROUND OF THE INVENTION

The idea of using bandages to protect a wound has been known for hundreds of years. More recently the use of an adhesive tape that includes a non-stick pad to allow easy removal and replacement has been popular. It is sold for commercial purchase at pharmacies and drug stores under the brandname “BAND AID”, or other names. Hospitals use a gauze (cotton) dressing held in place with “Surgical Tape” to cover the wound and absorb blood and wound emissions when a cut or wound is bleeding. The blood can seep into and be captured by the gauze.

During healing of an open wound or cut, the body forms a solid red ‘scab’ of clotted blood and lymph. Subsequent to scab formation, the body is able to heal the wound beneath the scab where the scab is in essence the bodies natural healing barrier to block ingress of pathogens. Unfortunately for wounds and cuts, the scab heals leaving a near permanent white scar and the larger, deeper, or longer the cut the larger will typically be the scab and as well, the scar. It would be an advantage to devise a superior method to 1) protect a wound during healing, 2) that would reduce the size of, or eliminate the resulting scar. and 3) it would also be an advantage to devise a superior method to reduce pain during healing and also 4) that would reduce the healing time.

One reason for this is that common adhesive BAND_AIDS and bandages with a gauze for fluid absorption successfully provide a covering over the wound, but they do not block bodily fluid flow out through the surface of the wounded skin. Further, for cuts, they do not compress opposing sides of the cut into one another. If flesh is cut, even deeply, and then it is compressed into itself, the flesh and skin surface will re-bond within minutes of the skin being cut. However, the tensile strength of the growing bond is initially almost zero. It takes several days for the bond strength to grow so that the wound is as strong as the skin was prior to the cut being made into the skin.

For cuts, especially for deep and or long cuts, another common practice is to suture, or stitch, the wound together. Sutures are better than an adhesive bandage because they compress the skin on one side of a cut line into the skin on the other side of a cut line. The suture forces the cut skin to close where the suture pressure is applied. Compared to just putting a gauze bandage over the cut line, a suture will achieve a superior closure including a much smaller scar.

Unfortunately, the scar formed, especially for large cuts such as are common during surgery, include a scar along the cut line and also scar dots where the suture and needle penetrated the skin to hold the opposite sides of the cut skin together. It would be advantageous if a new method for securely holding the skin on opposite sides of a wound could be devised to hold the skin on opposing sides of a cut into one another and to immobilize the opposing sides of a cut line in a proximal condition throughout the healing process.

Another benefit of sutures over band-aids is that they significantly close the wound for cuts, whether from surgery or from an accident. Closing the opposing sides of a wound reduces the area of internal body exposed to infectious microbes. But even sutures only compress the skin into itself at the locations of the stitches. Between the stitches, the flesh and skin pucker open and where each suture penetrates the skin twice to pull the cut together, the suture penetration provides another path into the interior of the body that microbes can follow.

It would be advantageous if there could be created a new technology that could entirely block the passage of microbes into the wound in part due to blocking access to the wound site, but also because the new technology could compress opposite sides of a cut line into one another without penetrating sutures. In this manner, one can imagine that a new technology might be able to do a superior job of compressing opposite sides of a cut into one another and at the same time, do so without requiring the addition of sutures and the resulting skin penetration holes where the suture penetrates into the body.

In another application, elderly people have thin and fragile skin. Often, sutures will cause the thin skin to tear, preventing their use. Applying band-aids do not promote rapid healing or avoid scars. It would be an advantage if a new method were devised that could more gently hold onto the fragile skin of elderly people and yet compress the opposing sides of a cut into one another.

In another application, people have sores that cover a small area of skin. These can come from for example, diabetes, or they could result from a fall to the ground where a knee becomes scraped and the skin over a larger area than a typical cut is grated and raw. Such a wound requires a protective barrier to block microbes from entering the wounded skin and to protect the sensitive area.

Washing and bathing can re-open sores and wounds and reverse the healing that was previously achieved. This can in some circumstances result in a situation where a sore on the skin persists virtually indefinitely. Sutures cannot close such a sore and typical bandages and band aids do not provide rapid healing.

A common problem with original adhesive bandages is that the tape material was not water vapor permeable. This resulted in the skin becoming overly hydrated, white, puffy and would not heal. When one has been in water for a long period of time, the skin becomes like dried “prunes”, all moist and wrinkly. Many adhesive bandages get around this problem by punching a number of holes through the water vapor impermeable tape. The water vapor coming from the body/skin can pass out through the holes in the tape and if there are sufficient holes, then the skin will not accumulate the water vapor and won't become overly hydrated and “prune” like. Applying this type of tape with holes directly over the cut or wound provides a path for blood to escape and microbes to enter. Rather than holes in the tape it would be advantageous if a tape that simply allowed water vapor to pass through were used.

With various bandages, a healing agent can often be applied to the wound and then the gauze covering can be applied over the wound with the healing agent and a surgical tape can hold the gauze with healing agent over the wound during healing. Various kinds of bandages are used and Band Aid is but one brand of numerous styles of adhesive bandages.

Most often, the healing agent is applied to the wound separately of the bandage. For a few bandage types, various healing agents including honey, and other typically anti microbial agents can be applied to the gauze that is then placed over the wound with the surgical tape holding the gauze impregnated with the healing agent, in place over the wound during the healing process. Unfortunately, this method fails to hold opposing sides of a cut together allowing for a larger scar to result. Antibacterial agents can be applied to the surface of “Healing Tape” as a thin stripe which will be over the cut or skin problem while healing.

In the case of open sores, it would be advantageous if a new healing tape could be created that would at once, provide a barrier to infectious microbes, a pathway for water vapor, and a healing agent that could persistently treat a chronic sore. In essence, it would be advantageous if a new sort of tape could be developed where the tape is in essence, a temporary replacement for skin that can remain in place for a week or two while a wound heals.

For this to work the tape would need to be somewhat liquid water resistant. In other words, if a cut is on the tip of a finger, then when the hands are washed whatever bandage is on the tip of the finger is going to get wet. Typical “Band Aid” style adhesive bandages wind up with the gauze becoming soaked with water and requiring replacement. Having a skin like tape that is sufficiently flexible and yet water resistant and water vapor permeable and able to remain affixed to the skin to protect a wound for a week during healing is an object of the present invention. This water resistant aspect is important for the device whether the wound happens to be a cut, tear, scrape, sore or other problem.

### BRIEF DESCRIPTION OF THE DRAWING FIGURES

FIG. 1 is a top plan view of a typical untreated cut or incision within skin, and also a partial section view taken along line A-A of the plan view of this figure.

FIG. 2 is a top plan view of a cut or incision which has been sutured closed, and including a partial section view taking along line B-B of this figure and also a partial section view taken along line C-C of this figure, section B-B located at a suture location and section C-C located at a midpoint between two sutures.

FIG. 3 is a perspective view of the healing tape of this invention being applied perpendicularly to a cut or incision within skin, the cut or incision indicated by a dashed line.

FIG. 4 Is a perspective view of the healing tape of this invention being applied parallel to a cut or incision within skin, the cut or incision indicated by a dashed line.

### DESCRIPTION OF HEALING TAPE

The healing tape is comprised of two components to heal cuts and incisions as well as other wounds and sores.

In FIG. 1, Plan View and Section AA, items 101 shows the two edges of a typical cut at the surface of skin before treatment; 102 shows the center and deepest extent of the cut, and item 103 shows the two opposing sides of the cut.

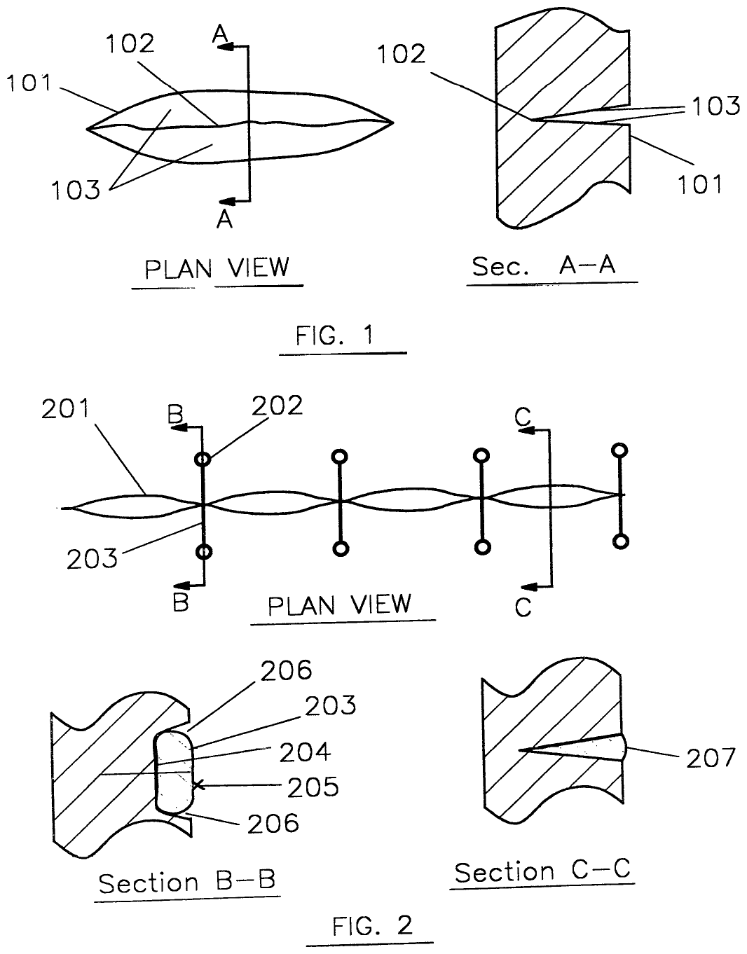

For comparison FIG. 2 shows prior and existing art, not the “healing tape” invention.

Present treatment of cuts and incisions with suturing is described using FIG. 2, Plan View and sections B-B and C-C.

In the Plan View, item 201 is the outside edge of a typical wound with typical sutures item 202 in place along the two edges of the cut and some distance from the edge of the cut. Section BB shows the suture thread as item 203 on the surface and 204, the same suture within or under the surface of the skin. Item 205 shows the required surgeons knot. Each suture is a separate unit and between any two sutures the skin, which is “rubbery”, is free to create an opening for blood escape and formation of scab materials, Item 207. Item 206 indicates the two openings in the surface of the skin for each suture created by the needle and pulled ‘open’ by tension in the suture thread. Scabs and scar tissue forms in each place the needle enters or exits, 206. Section C-C, item 207 shows the scab material which forms between each pair of sutures.

Item 301 is the surface of surrounding skin, 302 is the tape backing, 303 is the adhesive layer or adhesive surface. The healing tape composite, including tape backing, item 302, and adhesive 303, are applied directly to and over the cut while the two sides are pressed together.

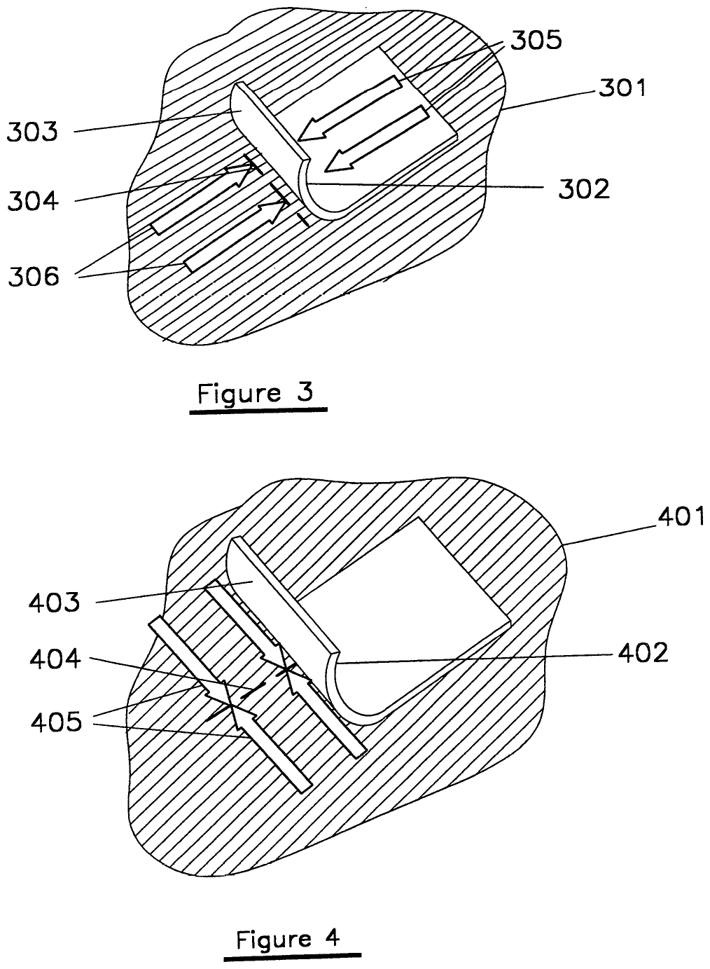

FIG. 3 and FIG. 4 show the components and typical application of the healing tape. FIG. 3 shows a cut item 304 shorter than the width of the tape, while FIG. 4 shows a longer cut with the healing tape applied lengthwise along the cut. The healing tape is comprised of a water vapor permeable tape backing (shown as items, 302 and 402) and an adhesive layer, item 303 and 403. The backing being water vapor permeable and somewhat water resistant has sufficient tensile strength to resist the relative motion or shearing of the skin, is bonded to the skin on opposite sides of a cut line during healing. The second surface, the adhesive layer, (items 303 and 403), with the backing are sufficiently flexible to adapt to the curved surface of the skin. The healing tape holds the two sides sides of the cut, 304, and 404 in compressive contact while capillaries can provide blood flow to all areas and surfaces of the cut, and carry away lymph fluid and blood that is depleted of oxygen and nutrients.

Application of the tape is performed in three steps.

For a short cut:

Step one, a length healing tape approximately 6 centimeters long, is placed on the skin on one side of the cut as shown in FIG. 3.

Step two requires applying pressure, arrows 305 on the cut using the adhered tape on one side and at the same time, pressure a short distance from the cut as shown by arrows 306 to force the two sides of the cut into intimate contact along its full length. Light finger pressure is adequate.

Step three requires the free end of the tape to be brought into contact with the skin while the two sides of the cut remain in intimate contact.

A longer cut, item 404, is addressed in similar fashion, the difference being the application of force and the need for the tape to be approximately 6 cm longer than the cut.

Step one—The tape is applied to the skin beyond one end of the cut.

Step two—force is applied to both sides of the cut, arrows items 405 before the tape is brought into contact with the skin where the two sides of the cut are in intimate contact. If the cut is quite long, the force and application of the tape is done in a series of identical steps.

Step three—the free end of the tape is brought into contact with the skin; a distance of about two centimeters.

The adhesive layer has sufficient bonding strength to hold opposite sides of the cut in intimate contact and to remain in place during the time required for healing. Small amounts of blood may initially escape the cut and accumulate under the healing tape. Initial bonding time for the two sides of the cut has been found to be approximately an hour or slightly more. Bond strength increases with time. Leaving the healing tape in place for a week to 10 days is recommended.

During this time the tape and the cut do not require any attention. Washing the area, including the tape is permissible. Heavy force or pressure on the tape are to be avoided for at least several days.

What I claim is:

1. A method for healing a cut using healing tape comprising the steps of:
wiping debris, if present, away from a cut,
cleansing the area around the cut with available materials, if any,
drying the cut if drying materials are available,
closing the cut flesh and skin so that the skin on opposite sides of the cut are brought back into proximity and contact
with the flesh on opposite sides of a cut moved into contact, cover the cut with healing tape so that with the skin on opposite sides of the cut are in contact
press the adhesive side of the tape to the skin on opposite sides of the cut
The cut becomes immobilized by the attachment of the healing tape as a result of the mechanical strength of the healing tape in tension and in shear.
The healing tape is comprised of;
a first surface comprised of a gas or water vapor permeable tape backing, the backing also being somewhat liquid water resistant and of sufficient tensile strength to resist the relative motion, and shearing, of the skin. And a second surface comprised of a skin compatible adhesive.
Results of using Healing Tape are:
Rapid reduction in pain; often to no pain as the tape is applied.
Rapid reduction in bleeding; often no bleeding or excess bleeding.
Elimination of scab formation if the two sides of the cut are in direct contact.
Reduction in infections; usually to no infections
Elimination of the need to redress wounds
Since no sutures are used, elimination of the need to remove sutures.
Elimination, or near elimination of scars; depending on care to close the wound.

2. The method of claim 1 where the opposite sides of the cut flesh are caused to close by applying the healing tape to a first side of a cut, then applying a tension to the tape so that the flesh is caused to close the cut, and then pressing the tape against the skin on the opposite side of the cut so that the tape is adhesively bonded to both sides of the cut. The tape will have immobilized the flesh on opposite sides of the cut.

3. The method of claim 1 where the water vapor permeability of the tape is larger than a value where skin, beneath the tape, becomes water saturated and whitish.

4. The method of claim 1 where the cut is optionally cleaned before the tape is applied.

5. The method of claim 1 where an anti-bacterial agent may be applied to the healing tape prior to its application to the skin.

6. The method of claim 1 where the tape is either cut from a continuous spool of tape or is obtained in precut lengths of any desired width.

7. The method of claim 1 where the cut results from surgery and the healing tape is applied to secure and close the wound post surgery.

8. The method of claim 1 where the opposite sides of the cut are pushed together with fingers prior to application of the healing tape.

9. A method of claim 1 where healing of a sore or other skin condition using a healing tape, the method comprised of,
cleansing the wound if possible
drying the wound and adjacent areas of skin where the healing tape will adhere
applying the healing tape so that it completely covers the wound
leaving the healing tape covering the wound for a period of at least 5 days
removing the healing tape, p1 the healing tape comprised of,
a first surface comprised of a gas permeable tape backing, the backing being water vapor permeable and liquid water resistant and of sufficient tensile strength to resist the relative motion, and shearing, unaffected skin, bonded to the tape. And a second surface comprised of a skin compatible adhesive

10. The method of claim 1 where the wound is comprised of scraped skin

11. The method of claim 1 where the wound is comprised of a surface sore including sores caused by diabetes.

12. The method of claim 1 where the wound is a tear.

13. The method of healing with Healing Tape where the wound is a void in the surface of the skin; the void being preferably less than one half inch in diameter and one eighth inch deep.
In this type of wound, there are no opposite surfaces to push into contact.
The steps of healing include:
Cleaning the void and the skin around the void if possible
Applying a strip of healing tape over the cut.
Leaving the tape in place for over much longer than one week, the length of time determined by the width and depth of the void. Healing a void is much slower than healing a cut or tear or even a sore. Healing tape has been used to heal voids; new skin material forms under the tape without scaffolding material being used.
In the observed use, new skin filled the void without leaving a scar.
